(19) United States
(12) Patent Application Publication
(10) Pub. No.: US 2021/0220072 A1
(43) Pub. Date: Jul. 22, 2021

(54) Environmental protection portable sharps box

(71) Applicant: Runhe JIANG, Nantong (CN)

(72) Inventor: Runhe JIANG, Nantong (CN)

(21) Appl. No.: 16/772,169

(22) PCT Filed: Dec. 27, 2018
(86) PCT No.: PCT/CN2018/124096

§ 371 Date: Jun. 12, 2020

(30) Foreign Application Priority Data
Dec. 24, 2018 (CN) 201811583422.0

(51) Int. Cl. A61B 50/36; C09D 5/16; C09D 123/06; C09D 7/61; B05D 1/02

(52) U.S. Cl. CPC A61B 50/362; C09D 5/1687; A61B 2050/0076; C09D 7/61; B05D 1/02; C09D 123/06

(57) ABSTRACT

The invention discloses an environmentally friendly portable sharps box. The main technical solutions include: a box body and a box cover, the outer edge of the open end of the box body has external threads, and the box cover has an L-shaped cross section A ring part, the inner edge of the vertical section of the ring part has an internal thread matched with the external thread, a ring groove is also formed on the box body, and a thread is formed between the bottom end of the box cover and the ring groove Connection, the box cover is provided with an input port, the box cover is provided with two semi-circular pressing blocks at the input port, and the box cover is provided with two semi-circular pressing blocks for sliding.

The invention relates to the field of medical waste treatment, more specifically, it relates to an environmentally friendly portable sharps box.

### BACKGROUND TECHNOLOGY

The sharps box is a medical storage box for collecting syringes, small glass products, blades, suture needles and other sharps. The sharps box is for single use. According to national requirements, it must be disposed of by medical waste disposal units within 24 hours Recycled and completely incinerated safely within 48 hours.

When the sharps box is used, the sharps such as used syringes, small glass products, blades, suture needles, etc. need to be discarded in the sharps box. Since the input port of the existing sharps box is often not effectively closed, When the sharps box accidentally causes overturning, the sharps in the sharps box often cause spillage, which causes medical pollution and has a great safety risk; in addition, due to the longer length of some syringes, the longer Direct placement of the sharps in the sharps box will take up more space, making the space for the sharps box into the sharps smaller, reducing the space usage of the sharps box.

### CONTENTS OF THE INVENTION

In view of the deficiencies in the prior art, the object of the present invention is to provide an environmentally friendly portable sharps box to solve the problems mentioned in the background art.

To achieve the above objectives, the present invention provides the following technical solutions:

An environmentally friendly portable sharps box includes a box body and a box cover. The outer edge of the open end of the box body has external threads. The box cover has a ring portion with an L-shaped cross section. The inner edge of the segment has an internal thread that cooperates with the external thread, a ring groove is also provided on the box body, a threaded connection is formed between the bottom end of the box cover and the ring groove, and an input There are two semicircular pressing blocks at the input port of the box cover, and two first sliding grooves for sliding the semicircular pressing blocks are opened on the box cover. A plurality of first springs are respectively connected between a chute groove wall and the two semicircular pressure blocks, and semicircular notches are provided on the two semicircular pressure blocks, and the two semicircles Cutting blades are respectively fixed at the notches on the shaped pressing blocks.

By adopting the above technical solution, thread locking between the box body and the box cover with two threads can strengthen the seal between the box body and the box cover and prevent liquid leakage; when the sharps box is in a normal state, the Under the action of the first spring in a chute, the two semi-circular pressure blocks will fit each other to close the input port on the lid of the sharps box, which can prevent the sharps box from tipping over The sharps inside spill out and cause pollution; when using sharps boxes to discard sharps, one of the semi-circular pressure blocks can be pulled to open a part of the input port on the box cover. Under the action of the first spring, the cutting blade at the semi-circular notch on the semi-circular pressure block cuts the sharp tool and shortens the sharp tool, so that the space utilization rate in the sharp box can be improved in disguise.

Further, one of the semi-circular pressing blocks is threadedly connected with a pull rod at the top, and the box cover is provided with a notch through which the pull rod passes.

By adopting the above technical solution, by pulling the rod rod connected to the semi-circular pressure block, the semi-circular pressure block can be driven to slide in the first chute, thereby facilitating the opening of the lid to discard the sharps. mouth.

Further, a blocking plate is clamped on the top of the box cover, a through slot for inserting sharps is formed at the center of the blocking plate, and a waist slot for the pull rod to pass through is also formed on the blocking plate.

By adopting the above technical solution, the blocking plate snapped on the top of the box cover can protect the sharps box from dropping the sharps at the box lid inlet, the sharps fall on the box cover, causing pollution to the box cover.

Further, a plurality of second chutes are opened inside the blocking plate, and a plurality of second chutes are slidably connected with an insertion block, and there is a connection between the insertion block and the bottom of the second chute groove In the second spring, a plurality of slots for inserting the insertion block are opened on the box cover.

By adopting the above technical solution, under the action of the second spring in the second chute, the insert block slidingly connected in the blocking plate will be inserted into the slot on the box cover, thereby clamping the blocking plate.

Further, the box body has a first fixing block integrally formed therewith, the first fixing block is detachably connected to a hanging rod through a plurality of first locking bolts, and the hanging rod has a convenient box body U-shaped hanging mouth.

By adopting the above technical solution, the sharps box can be conveniently hung on the shelf by using the hanging rod, which can effectively prevent the sharps box from overturning, and the U-shaped hanging opening on the hanging rod makes the hanging rod hanging more stable.

Further, the blocking plate has a stop ring integrally formed with the blocking plate on a side close to the semicircular pressure block, and the inner diameter of the blocking ring is the same as the radius of the through groove.

By adopting the above technical solution, the retaining ring can prevent the sharps from falling into the cavity between the blocking plate and the box lid input port.

Further, the box body has a second fixing block integrally formed therewith, a placement slot for label placement is opened in the second fixing block, and a second locking bolt is also screwed on the second fixing block.

By adopting the above technical solution, a label can be placed in the placement slot, which is convenient for the sharps investigator to understand the use status of the sharps box, and the position of the label in the placement slot can be conveniently locked by using the second locking bolt.

Further, the outer wall of the lid is coated with a layer of glitter paint, the glitter paint is prepared by the following method:

Take the following raw materials for each component by weight: 36-38 parts of vinyl resin, 22-24 parts of alkyd resin, 21-23 parts of urethane grade anhydrous butyl acetate, 11-13 parts of vinyl ester resin, amino 12-14 parts of resin, 8-10 parts of ethylenediaminetetraacetic acid, 6-8 parts of ethanol 5-8 parts of titanium dioxide, 1-3 parts of formamide, 12-14 parts of aluminum borosilicate, 6-10 parts of styrene Parts, 2-3 parts of cobalt isooctoate catalyst, 90-100 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 168-172° C. for 20-24 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed onto the surface of the lid with a spray gun.

Further, in the S1, the vinyl resin, alkyd resin, urethane grade anhydrous butyl acetate, vinyl ester resin, amino resin, ethylenediaminetetraacetic acid and ethanol are mixed uniformly and then added to the reaction kettle to maintain The internal temperature of the reactor was at 170° C. for 22 min.

Further, after the S4 spraying, the thickness of the flash paint on the outer surface of the lid is 0.4 mm.

By adopting the above technical solution, the flash paint on the box cover is more dazzling, which makes the position of the sharps box more conspicuous, and has a prompting effect for the sharps box user.

In summary, the present invention mainly has the following beneficial effects:

1. Thread locking between the box body and the box cover with two threads can strengthen the seal between the box body and the box cover and prevent liquid leakage;

2. When the sharps box is in the normal state, under the action of the first spring in the first chute, the two semi-circular pressure blocks will fit together to close the inlet at the lid of the sharps box It can prevent the sharps in the sharps box from spilling and causing pollution when the sharps box is overturned;

3. When using a sharps box to discard sharps, one of the semi-circular pressure pieces can be pulled to open a part of the input port on the lid. If the sharps are too long, the semi-circular pressure pieces can be loosened under the action of the first spring The cutting blade at the semi-circular notch on the semi-circular pressure block cuts the sharp object and shortens the sharp object, so that the space utilization rate in the sharp instrument box can be improved in disguise.

### FIGURE ILLUSTRATION

FIG. 1 is a structural cross-sectional view of an embodiment provided by the present invention;

FIG. 2 is an enlarged schematic structural view of part A in FIG. 1;

FIG. 3 is an enlarged schematic structural view of part B in FIG. 1;

FIG. 4 is an enlarged schematic structural view of part C in FIG. 1;

FIG. 5 is one of the structural schematic diagrams of an embodiment provided by the present invention;

FIG. 6 is a schematic diagram of an enlarged structure of the D part in FIG. 5;

FIG. 7 is an enlarged schematic view of the structure of E in FIG. 5

FIG. 8 is a second structural schematic diagram of an embodiment provided by the present invention;

FIG. 9 is an enlarged schematic view of the part F in FIG. 8.

In the picture: 1, box body; 2, box cover; 100, external thread; 21, ring part; 210, internal thread; 11, ring groove; 22, input port; 221, semicircular pressure block; 2210, first Chute; 2211, first spring; 2212, notch; 2213, cutting blade; 2215, tie rod; 23, notch; 24, blocking plate; 241, through groove; 242, waist groove; 243, second chute; 2431, insert block; 2432, second spring; 25, slot; 12, first fixing block; 121, first locking bolt; 122, hanging rod; 1221, U-shaped hanging mouth; 244, retaining ring; 13, Second fixing block; 131, placement slot; 132, second locking bolt.

### SPECIFIC IMPLEMENTATION MODALITIES

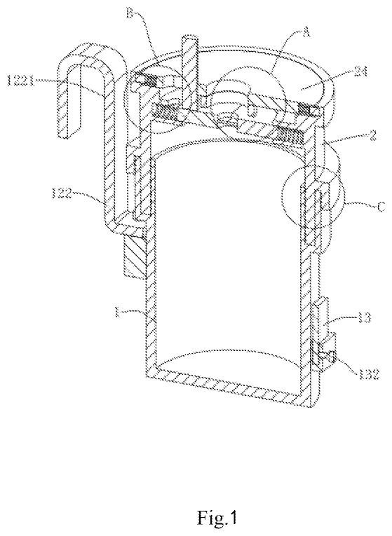

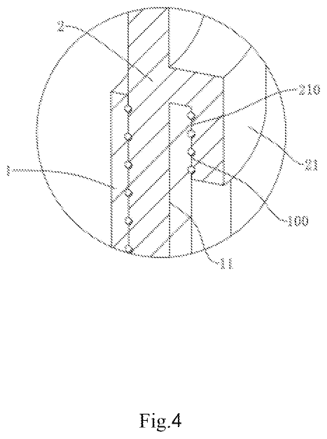

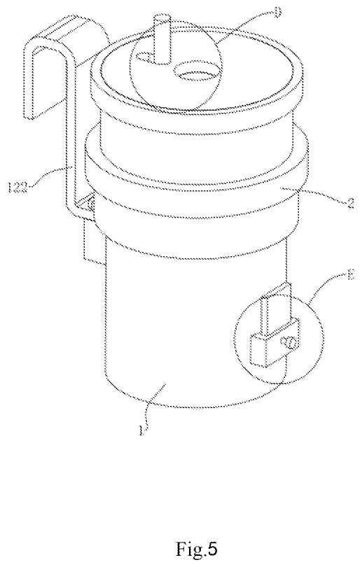

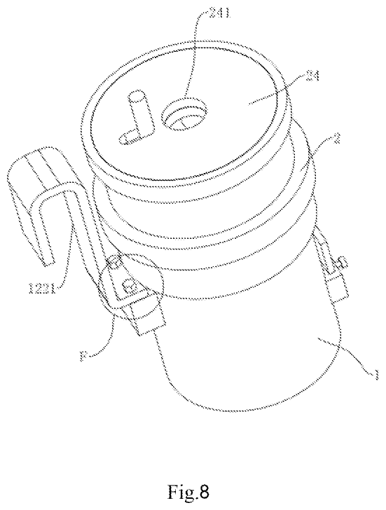

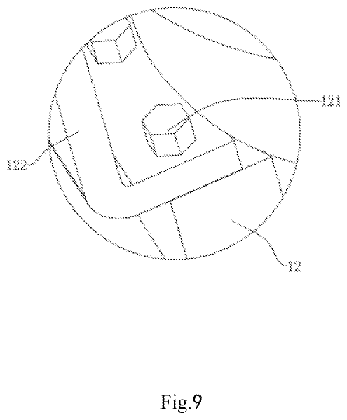

The present invention will be further described in detail below with reference to FIGS. 1-9.

### Example 1

1, 2, 3 and 4, an environmentally friendly portable sharps box includes a box body 1 and a box cover 2, wherein the outer edge of the open end of the box body 1 has an external thread 100 on the box cover 2 It has a ring portion 21 with an L-shaped cross section, wherein the inner edge of the vertical section of the ring portion 21 has an internal thread 210 that cooperates with the external thread 100, and a ring groove 11 is also formed on the box body 1 and the bottom of the box cover 2 Threaded connection between the end and the ring groove 11, when the box cover 2 is screwed outside the opening of the box body 1, the internal thread 210 at the ring portion 21 with an L-shaped cross section will be tightened with the external thread 100 on the outer wall of the box body 1, The part of the box cover 2 located in the ring groove 11 will be screwed with the ring groove 11 on the box body 1, that is, two threads are tightened at the same time to prevent liquid leakage; in order to facilitate the input of sharps, the box cover 2 is provided with The input port 22 is provided with two semi-circular pressing blocks 221 at the input port 22 of the box cover 2. The two semi-circular pressing blocks 221 are slidably connected to the input port 22 on the box cover 2 in the horizontal direction. Two first sliding grooves 2210 for sliding the semicircular pressing block 221 are provided on the box cover 2, and three first sliding grooves 2210 are connected between the groove wall of the first sliding groove 2210 and the two semicircular pressing blocks 221 respectively The spring 2211, under the action of the first spring 2211, the two semicircular pressing blocks 221 will abut each other, so that the sharps box can be closed as a whole; in order to cut the sharps, the two semicircular pressings Each block 221 is provided with a semi-circular notch 2212. A cutting blade 2213 is fixed at the notch 2212 of the two semi-circular pressing blocks 221 respectively. Both cutting blades 2213 are stainless steel blades, and the shape is Semi-circular; therefore, thread locking between the box body 1 and the box cover 2 with two threads can strengthen the seal between the box body 1 and the box cover 2 to prevent leakage; when the sharps box is in a normal state, Under the action of the first spring 2211 in the first chute 2210, the two semi-circular pressure pieces 221 will be attached to each other to close the input port 22 at the lid 2 of the sharps box, which can prevent sharp When the container is tipped, the sharps in the sharps box spill out and cause pollution; when the sharps box is used to discard the sharps, one of the semi-circular pressure blocks 221 can be pulled to open a part of the input port 22 on the lid 2 The sharp tool is too long, you can loose the semicircular pressure block 221, under the action of the first spring 2211, the cutting blade 2213 at the semicircular notch 2212 on the semicircular pressure block 221 cuts the sharp tool and cuts Short, so that the space utilization rate in the sharps box can be improved in disguise.

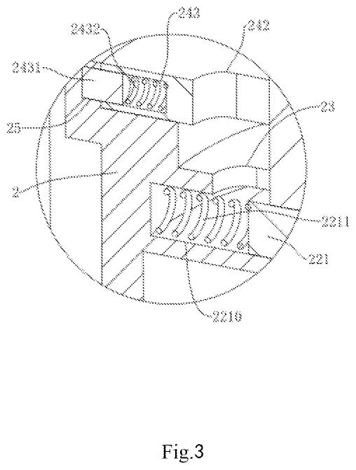

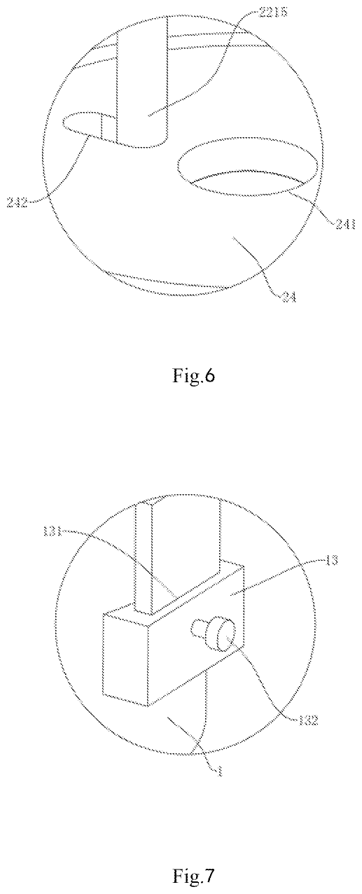

Referring to FIGS. 3 and 6, in order to control the semi-circular pressing block 221, a pull rod 2215 is threadedly connected to the top of one of the semi-circular pressing blocks 221, and a notch 23 for the pull rod 2215 to pass through is opened on the lid 2 Pulling the rod 2215 threaded on the semi-circular pressing block 221 moves, and can drive the semi-circular pressing block 221 to slide in the first sliding groove 2210, thereby facilitating opening the input port 22 on the box cover 2 for discarding sharps; In order to close the opening of the box cover 2, a blocking plate 24 is snapped on the top of the box cover 2, a through slot 241 is provided at the center of the blocking plate 24 to facilitate the insertion of sharps, and the blocking plate 24 is also provided with The waist groove 242 for the pull rod 2215 to pass through, and the blocking plate 24 snapped on the top of the lid 2 can protect the sharps box from dropping on the lid 2 when the sharps are dropped at the inlet 22 of the lid 2, Contaminate the lid 2.

Referring to FIGS. 1 and 3, in order to facilitate the installation of the blocking plate 24, two second sliding grooves 243 are opened inside the blocking plate 24, and both of the two sliding grooves 243 are slidably connected with an insert block 2431. A second spring 2432 is connected between the block 2431 and the bottom of the second chute 243, and two slots 25 for inserting the insertion block 2431 are opened on the cover 2, and the second spring 2432 is in the second chute 243 Under the action of, the plug block 2431 slidingly connected in the blocking plate 24 will be inserted into the slot 25 on the box cover 2, thereby clamping and fixing the blocking plate 24.

8 and 9, since the traditional sharps box is placed on the table or the ground, it is prone to collision and overturning. In order to prevent the sharps box from colliding and overturning, the box body 1 has a first fixing block integrally formed therewith 12, and the first fixing block 12 is disassembled and connected to the hanging rod 122 through two first locking bolts 121, wherein the hanging rod 122 has a U-shaped hanging opening 1221 for hanging the box body 1 by using the hanging rod 122 can conveniently hang the sharps box on the shelf, which can effectively prevent the sharps box from overturning, and the U-shaped hanging opening 1221 on the hanging rod 122 makes the hanging rod 122 more stable.

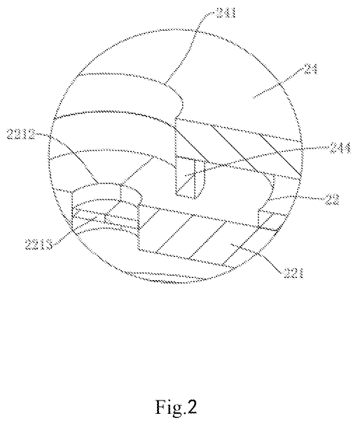

Referring to FIGS. 1 and 2, at the same time, the blocking plate 24 has a stop ring 244 integrally formed with the blocking plate 24 on the side close to the semicircular pressing block 221, wherein the inner diameter of the stop ring 244 is the same as the radius of the through groove 241, 244 can prevent the sharps from falling into the cavity between the blocking plate 24 and the inlet 22 of the lid 2.

5 and 7, in order to mark the sharps box, the box body 1 has a second fixing block 13 integrally formed therewith, and a placement slot 131 for placing a label is opened in the second fixing block 13, and The second fixing block 13 is also threadedly connected with a second locking bolt 132, and a label can be placed in the placement slot 131, which is convenient for sharps investigators to understand the use status of the sharps box, and the second locking bolt 132 can be conveniently used The position of the label in the placement slot 131 is locked.

### Example 2

The difference from Embodiment 1 is that the outer wall of the box cover 2 is coated with a layer of glitter paint. According to national standards, the box body 1 of the sharps box needs to be eye-catching, so the box body 1 of the sharps box is generally orange or red In order to make the sharps box more eye-catching, the outer wall of the box cover 2 is also coated with a layer of glitter paint, where the glitter paint is prepared by the following method:

Take the following raw materials for each component by weight: 36 parts of vinyl resin, 22 parts of alkyd resin, 21 parts of urethane grade anhydrous butyl acetate, 11 parts of vinyl ester resin, 12 parts of amino resin, ethylenediamine tetra 8 parts of acetic acid, 6 parts of ethanol, 5 parts of titanium dioxide, 1 part of formamide, 12 parts of aluminum borosilicate, 6 parts of styrene, 2 parts of cobalt isooctanoate catalyst, and 90 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 3

The difference from Example 2 is the preparation of the flash paint on the surface of the lid 2, wherein the preparation method of the flash paint is as follows:

Take the following raw materials for each component by weight: 37 parts of vinyl resin, 23 parts of alkyd resin, 22 parts of urethane grade anhydrous butyl acetate, 12 parts of vinyl ester resin, 12 parts of amino resin, ethylenediamine tetra 8 parts of acetic acid, 6 parts of ethanol, 5 parts of titanium dioxide, 1 part of formamide, 12 parts of aluminum borosilicate, 6 parts of styrene, 2 parts of cobalt isooctoate catalyst, and 90 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 4

The difference from Example 2 is the preparation of the flash paint on the surface of the lid 2, wherein the preparation method of the flash paint is as follows:

Take the following raw materials for each component by weight: 37 parts of vinyl resin, 23 parts of alkyd resin, 22 parts of urethane grade anhydrous butyl acetate, 12 parts of vinyl ester resin, 13 parts of amino resin, ethylenediamine tetra 9 parts of acetic acid, 7 parts of ethanol, 6 parts of titanium dioxide, 1 part of formamide, 12 parts of aluminum borosilicate, 6 parts of styrene, 2 parts of cobalt isooctanoate catalyst, and 90 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 5

The difference from Example 2 is the preparation of the flash paint on the surface of the lid 2, wherein the preparation method of the flash paint is as follows:

Take the following raw materials for each component by weight: 37 parts of vinyl resin, 23 parts of alkyd resin, 22 parts of urethane grade anhydrous butyl acetate, 12 parts of vinyl ester resin, 13 parts of amino resin, ethylenediamine tetra 9 parts of acetic acid, 7 parts of ethanol, 6 parts of titanium dioxide, 2 parts of formamide, 13 parts of aluminum borosilicate, 8 parts of styrene, 2 parts of cobalt isooctoate catalyst, and 95 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 6

The difference from Example 2 is the preparation of the flash paint on the surface of the lid 2, wherein the preparation method of the flash paint is as follows:

Take the following raw materials for each component by weight: 38 parts of vinyl resin, 24 parts of alkyd resin, 23 parts of urethane grade anhydrous butyl acetate, 13 parts of vinyl ester resin, 14 parts of amino resin, ethylenediamine tetra 9 parts of acetic acid, 7 parts of ethanol, 6 parts of titanium dioxide, 2 parts of formamide, 13 parts of aluminum borosilicate, 8 parts of styrene, 2 parts of cobalt isooctoate catalyst, and 95 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 7

The difference from Example 2 is the preparation of the flash paint on the surface of the lid 2, wherein the preparation method of the flash paint is as follows:

Take the following raw materials for each component by weight: 38 parts of vinyl resin, 24 parts of alkyd resin, 23 parts of urethane grade anhydrous butyl acetate, 13 parts of vinyl ester resin, 14 parts of amino resin, ethylenediamine tetra 10 parts of acetic acid, 8 parts of ethanol, 8 parts of titanium dioxide, 3 parts of formamide, 13 parts of aluminum borosilicate, 8 parts of styrene, 2 parts of cobalt isooctanoate catalyst, and 95 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

### Example 8

The difference from Example 2 is the preparation of the flash paint on the surface of the lid 2, wherein the preparation method of the flash paint is as follows:

Take the following raw materials for each component by weight: 38 parts of vinyl resin, 24 parts of alkyd resin, 23 parts of urethane grade anhydrous butyl acetate, 13 parts of vinyl ester resin, 14 parts of amino resin, ethylenediamine tetra 10 parts of acetic acid, 8 parts of ethanol, 8 parts of titanium dioxide, 3 parts of formamide, 14 parts of aluminum borosilicate, 10 parts of styrene, 3 parts of cobalt isooctanoate catalyst, and 100 parts of water;

S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 170° C. for 22 min;

S2. Preparation of the second pre-preparation liquid: mixing titanium dioxide, formamide, aluminum borosilicate, styrene, cobalt isooctanoate catalyst and water, and stirring them evenly;

S3. Preparation of flash paint: add the second pre-preparation liquid obtained in S2 to the first pre-preparation liquid in the reaction kettle, mix thoroughly and cool to room temperature to obtain a flash paint;

S4. Spraying: The flash paint obtained in S3 is evenly sprayed on the surface of the lid 2 with a spray gun.

Further, after the S4 spraying, the thickness of the flash paint on the outer surface of the box cover 2 is 0.4 mm.

Since the flash paint on the box cover 2 is more dazzling, the position of the sharps box is more conspicuous, which has a prompting effect for the sharps box user.

The case cover 2 coated with the glitter paint in Examples 2-8 was tested for surface hardness and peel strength. For comparison, the data of all examples are normalized based on the data of Example 2.

### Specification

TABLE 1

| | Surface hardness | Peel strength |
|---|---|---|
| Example 2 | 100% | 100% |
| Example 3 | 99% | 98% |
| Example 4 | 99% | 98% |
| Example 5 | 103% | 105% |
| Example 6 | 101% | 102% |
| Example 7 | 98% | 97% |
| Example 8 | 99% | 98% |

It can be seen from the above that since the test results of the surface hardness and peel strength of the lid 2 in Example 5 are higher than those of the lid 2 coated with the glitter paint in other embodiments, the preparation of the glitter paint given in Example 5 The method is the best choice.

This specific embodiment is merely an explanation of the present invention, and it is not a limitation of the present invention. Those skilled in the art can make modifications without creative contribution to this embodiment after reading this specification, as long as they have rights in the present invention Within the scope of the requirements are protected by patent law.

1. An environmentally friendly portable sharps box, including a box body (1) and a box cover (2), characterized in that: the outer edge of the open end of the box body (1) has external threads (100) (2) A ring portion (21) with an L-shaped cross section is provided on the inner edge of the vertical section of the ring portion (21), which has an internal thread (210) matching the external thread (100) The body (1) is also provided with a ring groove (11), the bottom end of the box cover (2) is screwed between the ring groove (11), and the box cover (2) is provided with an input port (22), two semicircular pressing blocks (221) are provided at the input port (22) of the box cover (2), and two semicircular openings are provided on the box cover (2) A first sliding groove (2210) for sliding the pressing block (221), and a plurality of first springs are respectively connected between the groove wall of the first sliding groove (2210) and the two semicircular pressing blocks (221) (2211), two semicircular pressing blocks (221) are provided with semicircular notches (2212), and the notch (2212) on the two semicircular pressing blocks (221) The cutting blades (2213) are fixed respectively.

2. An environmentally friendly portable sharps box according to claim 1, characterized in that one of the semi-circular pressing blocks (221) is threadedly connected with a pull rod (2215) at the top, and the box cover (2) is A notch (23) is provided for the pull rod (2215) to pass through.

3. An environmentally friendly portable sharps box according to claim 2, characterized in that: a blocking plate (24) is snapped on the top of the lid (2), and the blocking plate (24) is provided with A through-groove (241) for convenient insertion of sharps, a waist-shaped groove (242) through which the pull rod (2215) passes is also formed on the blocking plate (24).

4. An environmentally friendly portable sharps box according to claim 3, wherein the blocking plate (24) is provided with a plurality of second slide grooves (243), and a plurality of the second slide grooves (4) Inserts (2431) are slidingly connected inside, and a second spring (2432) and the box cover (2) are connected between the inserts (2431) and the bottom of the second slide groove (243) The upper opening is provided with a plurality of slots (25) for inserting the insertion block (2431).

5. An environmentally friendly portable sharps box according to claim 1, characterized in that: the box body (1) has a first fixing block (12) integrally formed therewith, and the first fixing block (12) Is connected to the hanging rod (122) through several first locking bolts (121), and the hanging rod (122) has a U-shaped hanging port (1221) for facilitating the hanging of the box body (1).

6. An environmentally friendly portable sharps box according to claim 3, characterized in that: the blocking plate (24) is integrated with the blocking plate (24) on the side close to the semicircular pressing block (221) A shaped stop ring (244), the inner diameter of the stop ring (244) is the same as the radius of the through groove (241).

7. An environmentally friendly portable sharps box according to claim 1, characterized in that: the box body (1) has a second fixing block (13) integrally formed therewith, the second fixing block (13) A placing groove (131) for placing a label is opened, and a second locking bolt (132) is also screwed on the second fixing block (13).

8. An environmentally friendly portable sharps box according to claim 1, characterized in that the outer wall of the box cover (2) is also coated with a layer of glitter paint, which is prepared by the following method:
Take the following raw materials for each component by weight: 36-38 parts of vinyl resin, 22-24 parts of alkyd resin, 21-23 parts of urethane grade anhydrous butyl acetate, 11-13 parts of vinyl ester resin, amino 12-14 parts of resin, 8-10 parts of ethylenediaminetetraacetic acid, 6-8 parts of ethanol, 5-8 parts of titanium dioxide, 1-3 parts of formamide, 12-14 parts of aluminum borosilicate, 6-10 parts of styrene Parts, 2-3 parts of cobalt isooctoate catalyst, 90-100 parts of water;
S1. Preparation of the first pre-preparation liquid: mix vinyl resin, alkyd resin, urethane grade anhydrous butyl acetate, vinyl ester resin, amino resin, ethylenediaminetetraacetic acid and ethanol, and evenly add them to the reactor, Maintain the internal temperature of the reactor at 168-172° C. for 20-24 min;
S2. Preparation of the second pre-preparation liquid: mixing titanium dioxide, formamide, aluminum borosilicate, styrene, cobalt isooctanoate catalyst and water, and stirring them evenly;
S3. Preparation of flash paint: add the second pre-preparation liquid obtained in S2 to the first pre-preparation liquid in the reaction kettle, mix thoroughly and cool to room temperature to obtain a flash paint;
S4. Spraying: The flash paint obtained in 83 is evenly sprayed on the surface of the box cover (2) with a spray gun.

9. The method for preparing an anti-fouling coating according to claim 8, characterized in that: in S1, vinyl resin, alkyd resin, urethane grade anhydrous butyl acetate, vinyl ester resin, amino resin And ethylenediaminetetraacetic acid and ethanol are mixed evenly and then added to the reactor, keeping the internal temperature of the reactor at 170° C. for 22 min.

10. The method for preparing an antifouling coating according to claim 8, wherein after the S4 spraying, the thickness of the flash paint on the outer surface of the lid (2) is 0.4 mm.
